(19) United States
(12) Patent Application Publication
(10) Pub. No.: US 2026/0002933 A1
(43) Pub. Date: Jan. 1, 2026

(54) PROTEIN DETECTION METHOD AND DETECTION DEVICE AS WELL AS USE

(71) Applicant: SUZHOU INSTITUTE OF NANO-TECH AND NANO-BIONICS (SINANO) , CHINESE ACADEMY OF SCIENCES, Suzhou (CN)

(72) Inventors: Jiong LI, Suzhou (CN); Yiran HE, Suzhou (CN); Kefeng PU, Suzhou (CN); Chaoxu LUO, Suzhou (CN); Xiaodong MA, Suzhou (CN); Min JIANG, Suzhou (CN)

(73) Assignee: SUZHOU INSTITUTE OF NANO-TECH AND NANO-BIONICS (SINANO) , CHINESE ACADEMY OF SCIENCES, Suzhou (CN)

(21) Appl. No.: 18/878,880

(22) PCT Filed: Jun. 5, 2024
(86) PCT No.: PCT/CN2024/097540

§ 371 Date: Dec. 25, 2024

(30) Foreign Application Priority Data
Dec. 5, 2023 (CN) 202311657451.8

(51) Int. Cl. G01N 33/543; G01N 33/533; G01N 33/68

(52) U.S. Cl. CPC G01N 33/54306; G01N 33/533; G01N 33/6854

(57) ABSTRACT

A protein detection method and a detection device as well as use are provided. The protein detection method includes: fixing a probe type graphic encoding chip coupled with a specific capture antibody in the detection device, and meanwhile respectively sealing SAPE and a detection antibody in the detection device; and when in detection, performing detection and analysis based on an enzyme linked immunosorbent assay (ELISA) principle through the probe-type graphic encoding chip after the detection antibody and the SAPE are respectively dissolved, so as to obtain protein quantitative analysis results. In the protein detection method, the adopted Lab-in-Tip technology can complete processes such as sampling, washing, detection antibody hybridization, washing and binding of fluorescence labeled streptavidin and a biotin labeled detection antibody in such the Lab-in-Tip device, and has the advantages of multiplexed detection, such as quick completion, convenience, high flexibility, good specificity, few sample capacity and wide detection range.

### CROSS REFERENCE TO THE RELATED APPLICATIONS

This application is the national phase entry of International Application No. PCT/CN2024/097540, filed on Jun. 5, 2024, which is based upon and claims priority to Chinese Patent Application No. 202311657451.8, filed on Dec. 5, 2023, the entire contents of which are incorporated herein by reference.

### TECHNICAL FIELD

The present disclosure relates to the technical field of biological detection, and particularly to a protein detection method and a detection device as well as use.

### BACKGROUND

Multiplexed immunoassay has been widely applied to clinical diagnosis, therapeutics, drug discovery and clinical proteomic research. As there are hundreds of protein biomarkers used for clinical and pharmaceutical applications, it is required to use time-saving and cost-effective analysis strategy-multiplexed immunoassay. A liquid chip technology, referred to as a suspension array technology (SAT), is a new high-throughput biological chip technology combined with flow cytometry, a laser technology, fluidics and the like, which can be used for protein and nucleic acid detection. Compared with the defects in the traditional enzyme linked immunosorbent assay (ELISA) technology, such as poor repeatability, easy false positivity and time and energy wasting, a new suspension array is wide in linear range and convenient to operate, and can conduct efficient multiplexed detection.

A microsphere suspension chip is a new biological chip technology platform based on Luminex xMAP technology. The Luminex xMAP technology, as the earliest biological chip technology that is certified by the U.S. Food and Drug Administration (FDA) and used for clinical diagnosis, has become one of multiplexed detection technologies widely used. It can achieve qualitative and quantitative purposes by jointly detecting microsphere encoding and reporting fluorescence via red and green laser beams, and therefore is a new-generation high-throughput molecule detection technology platform following gene chips and protein chips.

However, the Luminex's xMAP technology also has some defects, for example, since there are many and complicated contents in serum, when an antibody is detected, a detection background signal is too high so that a signal to noise ratio is reduced, and the reliability of experimental results is lowered. Furthermore, although the Luminex company has made great efforts in terms of continuously improving detection accuracy, enhancing the performance of air compressors, increasing detection weight and flux and the like for the past few years in order to optimize microsphere and detection platforms, it inevitably brings higher technological complexity and high costs.

A graph is another popular code system of a suspension array, which uses a group of visually distinguishable patterns such as embedded barcodes or physical shapes to identify different analytical particles. Similar to the xMAP system based on particles, the graphical suspension array belongs to pseudo homogeneity determination with near-solution diffusion kinetics, resulting in higher mixing efficiency. Importantly, graphic encoding particles have unique characteristics, which can overcome the defects of color encoding beads, such as better particle shape/size consistency, digital and analog decoding process, and bigger flexibility for selection of materials with different chemical, mechanical and/or optical properties for customized particles.

So far, most of the proposed graphical suspension arrays focus on multiplex detection of nucleic acids, with less work on immunoassay of protein analytes, which is partially because of complex analysis development issues such as analyte fragility, reagent reproducibility and non-specific binding. A review of the existing publications in this field indicates that only a few works have proposed feasible methods for protein determination of LOD (limit of detection) at 1 pg/mL level or higher; moreover, in the protein quantitative analysis based on ELISA principle using a suspension array, since the suspension chip is not fixed in an array mode, the washing process needs long-term natural subsidence or multiple centrifugations in the reaction stage before multiplexed detection, such the process inevitably leads to greatly prolonged overall reaction time and wasted time and labor, and has the disadvantages of poor repeatability, easy occurrence of false positivity, time and labor wasting, and the like.

### SUMMARY

In view of the technical problems existing in human serum protein analysis using the existing technology, such as poor repeatability, easy false positivity and time and energy wasting, the present disclosure provides a protein detection method and detection device based on a Lab-in-Tip technology, and use thereof. The protein detection method has the advantages of good repeatability, simple detection device, short detection time and the like.

In order to achieve the above objective, the present disclosure provides the following technical solution: a protein detection method, comprising: fixing a probe type graphic encoding chip coupled with a specific capture antibody in a detection device, meanwhile respectively sealing SAPE (R-phycoerythrin labeled streptavidin) and a detection antibody in the detection device; during the detection, respectively dissolving the detection antibody and the SAPE, or respectively preparing an SAPE solution and a detection antibody solution in advance, then performing detection and analysis through the probe type graphic encoding chip based on ELISA principle, so as to obtain protein quantitative analysis results.

In some embodiments, the detection device is based on an Lab-in-Tip technology, and specifically comprises a detection tube and a storage tube; the probe type graphic encoding chip coupled with the specific capture antibody is embedded in the detection tube; the SAPE and the detection antibody are embedded in the inner wall of the storage tube; or a detection antibody solution and an SAPE solution are directly prepared and placed in a pipette workstation; to-be-detected samples are placed in the pipette workstation, the volume of a solvent entering the storage tube is controlled, and the detection antibody and the SAPE enter the detection tube after being respectively dissolved in turn; or the detection antibody solution and the SAPE solution are directly prepared and directly transferred into the detection tube, so as to achieve protein quantitative detection and analysis.

In some embodiments, the probe type graphic encoding chip comprises: after the graphic encoding chip is subjected to surface modification, a probe molecule is coupled to the graphic encoding chip to obtain the probe type graphic encoding chip.

In some embodiments, the probe molecule is a specific capture antibody.

In some embodiments, the graphic encoding chip is an encoding suspension chip based on silica particles.

In some embodiments, a method for preparing the probe type graphic encoding chip comprises the following steps:
(1) dispersing an encoding suspension chip based on silica into an ethanol solution of amino polydimethylsiloxane (APDMS) for reaction, so that the encoding suspension chip is subjected to amino surface modification to obtain the amino modified encoding suspension chip;
(2) dispersing the amino modified encoding suspension chip obtained after reaction in step (1) into a succinic anhydride solution, and then performing vibration reaction at a room temperature for surface modification, so as to further obtain the carboxyl modified graphic encoding chip; and
(3) performing an activation reaction on the carboxyl modified graphic encoding chip obtained in step (2), and then performing coupling reaction on the activated graphic encoding chip and a probe molecule solution to couple the probe molecule to the surface of the graphic encoding chip so as to obtain the probe type graphic encoding chip.

Further, in step (3), the activation reaction is carried out at a room temperature for 20-40 min; the activation solution is a 4-methylmorpholine sulfonic acid (MES) buffer solution containing 1-ethyl (3-dimethylaminopropyl) carbodiimide (EDC) and N-hydroxysuccinimide (NHS).

In some embodiments, the coupling reaction is carried out at 0-4° C. for 6 h-12 h;

In some embodiments, the probe molecule solution is a NaAc-HAc buffer solution of a probe molecule.

In some embodiments, the probe type graphic encoding chip coupled with the specific capture probe is embedded in the detection device based on the Lab-in-Tip technology; the detection antibody solution and the SAPE solution respectively enter the detection device in turn so as to achieve protein quantitative detection and analysis through the probe type graphic encoding chip.

In some embodiments, the detection device at least comprises a detection tube and a storage tube, and the probe type graphic encoding chip is embedded in the detection tube.

In some embodiments, the detection antibody and the SAPE are embedded in the inner wall of the storage tube, enter the detection tube after being blown and dissolved by the pipette workstation to react with the graphic encoding chip; or, the detection antibody the SAPE are dissolved to be directly prepared into solutions, and then contact and react with the graphic encoding chip in turn in the detection tube under the blowing action of the pipette or the pipette workstation so as to achieve protein quantitative detection and analysis.

Specifically, the protein detection method based on the Lab-in-Tip technology comprises the following steps:
S1, fixing a probe type graphic encoding chip coupled with a specific capture antibody in the inner wall of the detection tube in the detection device through natural subsidence;
S2, preparing a detection antibody solution and/or an SAPE solution, or embedding the detection antibody and/or the SAPE in the detection device;
S3, adding a to-be-detected protein sample solution into a sample well plate, and putting the well plate in the pipette workstation;
S4, connecting the detection device with the pipette workstation or the pipette;
S5, putting a well plate distributed with a phosphate buffer solution into the pipette workstation, dissolving the detection antibody embedded in the detection device to form a solution, or, placing the prepared detection antibody solution into the well plate, performing a solution blowing action through the pipette or the pipette workstation so that the detection antibody solution reacts with the graphic encoding chip; and washing after the reaction is ended;
S6, repeating the operation in the above step, putting the prepared SAPE solution into the detection device, or, dissolving the SAPE embedded in the detection device so that the SAPE reacts with the probe type graphic encoding chip; and washing after the reaction is ended; and
S7, taking out the detection tube for image data collection, and performing qualitative analysis or quantitative analysis treatment on the probe type graphic encoding chip in the detection tube to obtain determination results.

In order to achieve another objective, the present disclosure also provides a detection device based on a Lab-in-Tip technology applied to the above protein detection.

In some embodiments, the detection device comprises a detection tube and a storage tube which are detachably connected.

Further, the storage tube has a pipette sucker structure based on the Lab-in-Tip technology.

Further, the storage tube is in a cone-shaped structure, and comprises a tip part and a tail part; the tip part is connected with the detection tube; the tail part is connected with the pipette or the pipette workstation.

Further, the probe type graphic encoding chip is fixed in the detection device.

In some embodiments, the detection antibody and/or the SAPE enters the detection tube to react with the probe type graphic encoding chip.

In some embodiments, the detection antibody and the SAPE are directly lyophilized on the surface of the inner wall of the storage tube through a lyophilization method. A solution flows through the surface, so that the detection antibody and the SAPE are respectively dissolved.

In some embodiments, the detection antibody and the SAPE are lyophilized at different positions, the detection antibody is sealed on the surface of the storage tube close to the inner wall of one end of the detection tube, and the SAPE is sealed at the rear end close to the cone-shaped sucker of the storage tube.

Preferably, the probe type graphic encoding chip is embedded in the detection tube.

As a preferred embodiment, the detection antibody and the SAPE can be sealed on the surface of the inner wall of the storage tube by using a lyophilization method, can enter the detection tube after being dissolved and react with the specific capture antibody coupled with the surface of the probe type graphic encoding chip.

As another preferred embodiment, the detection antibody and the SAPE are respectively prepared into solutions in advance, and then transferred to the detection tube and react with the specific capture antibody coupled with the surface of the probe type graphic encoding chip.

Further, the storage portion comprises a first storage area and a second storage area. Further, the first storage area is arranged between the tip part and ½ of the storage tube, and the detection antibody is sealed on the inner wall of the first storage area.

Further, the second storage area is arranged between the tail part and ½ of the storage tube, and the SAPE is sealed on the inner wall of the second storage tube.

Further, the storage tube and the detection tube are connected through a connection member, or directly connected without the connection member.

Further, the direct connection arrangement comprises: the storage tube and the detection tube are connected through a threaded part, a fastener, a seal or other connection manners, or devices that can achieve the sealing and connection of the storage tube and the detection tube in the prior art are used, which are all included within the scope of protection of the present disclosure.

Further, the storage tube and the detection tube are connected through the connection member, one end of the connection member is connected with the tip of the storage tube, and the other end of the connection member is connected with the detection tube.

Further, the connection member is one of a latex tube, a rubber tube, a thermoplastic tube or an ultraviolet (UV) hose, which can seal and connect the storage tube and the detection tube.

Further, the detection tube comprises any one of a capillary tube, a plastic tube, a quartz tube and a glass tube, which can fix the surface modified probe type graphic encoding chip on the inner wall of the storage tube through a natural subsidence method.

Further, a biotinylated detection antibody mixture and SAPE are fixed on the inner wall of the pipette tube in turn.

The use in protein quantitative analysis based on ELISA principle can be achieved by using the above protein detection method based on the Lab-in-Tip technology, especially, the probe type graphic encoding chip used in the Lab-in-Tip technology provides a 128-plexed encoding spaces, so as to complete 128-plexed detection in a single pipette sucker, thereby achieving the sensitivity of less than 1 pg/ml within the reaction time of 1 h.

The technical solution of the present disclosure has the beneficial effects:

1. Through adoption of the technical solution of the present disclosure, the used Lab-in-Tip technology can complete processes such as sampling, washing, detection antibody hybridization, washing and binding of fluorescence labeled streptavidin and a biotin labeled detection antibody in one pipette sucker, and therefore has the advantages of multiplexed detection, such as quick completion, convenience, high flexibility, good specificity, few sample capacity and wide detection range.

2. Through adoption of the technical solution of the present disclosure, the probe type graphic encoding chip used in the Lab-in-Tip technology provides 128-plexed encoding spaces so as to complete 128-plexed detection in the single pipette sucker, thereby greatly reducing the sample size required in the reaction process, significantly shortening the time of washing steps, performing multiplexed detection which is quick, convenient, high in throughput, few in sample size, good in repeatability, high in sensitivity and wide in linear range, and achieving the sensitivity of less than 1 pg/mL within the reaction time of 1 h.

3. Through adoption of the technical solution of the present disclosure, the detection device based on the Lab-in-Tip technology can be assembled by using the conventional experiment devices in a lab. The pipette sucker is simply modified so as to protein quantitative detection in the detection device. The detection device is simple in structure and easy to assemble, the assembled components are only selected conventional devices in the lab, such as the pipette sucker, the silicone tube and the capillary tube. Furthermore, protein detection is simple in process and correct in result, especially, the detection time is greatly shortened.

### BRIEF DESCRIPTION OF THE DRAWINGS

FIG. 1 is a structural diagram of a Lab-in-Tip device provided in example 1 of the present disclosure.

FIG. 2 is a diagram of a graphic encoding chip provided in example 1 of the present disclosure.

FIG. 3 is a performance standard curve graph of IL-8 within the reaction time of 1 h in example 1 of the present disclosure.

FIGS. 4A-4M are performance standard curve graphs of IL-1β, IL-2, IL-4, IL-5, IL-6, IL-8, IL-10, IL-12, IL-17, TNF-α, IFN-α, IFN-γ and GM-CSF within the reaction time of 1 h for 13-plexed detection.

FIG. 5 is a performance standard curve graph of IL-8 after being stored for 3 months at 4° C. for stability detection in example 1 of the present disclosure.

FIGS. 6A-6D are performance standard curve graphs for 4-plexed detection when the total reaction volume is only 10 μL in example 4 of the present disclosure.

FIGS. 7A-7C are performance standard curve graphs for 3-plexed detection within the reaction time of 15 min in example 5 of the present disclosure.

### DETAILED DESCRIPTION OF THE EMBODIMENTS

To make the purpose, technical solution and advantages of the embodiments of the present disclosure more clear, the technical solution in the embodiments of the present disclosure will be clearly and completely described, obviously, the described embodiments are only some embodiments of the present disclosure, but not all the embodiments. Based on the embodiments of the present disclosure, other embodiments obtained by persons of ordinary skill in the art without creative efforts are all included within the scope of protection of the present disclosure.

The publications of all patent and non-patent literatures referred in the present disclosure are incorporated herein by reference.

The terms “comprise”, “include”, “contain”, “cover”, “have”, “carry” or their any other variations are all intended to encompass non-exclusive inclusions. For example, the process, method, product, or equipment that includes the list of elements is not necessarily limited to those elements, but may include other elements that are not explicitly listed or inherent to the process, method, product, or equipment. In addition, unless otherwise specified, “or” refers to inclusive “or” rather than exclusive “or”. For example, condition A or B satisfies any of the following: A is true (or existing) and B is false (or non-existent), A is false (or non-existent) and B is true (or existing), and both A and B are true (or existing).

The phrase “one or more” is intended to encompass non-exclusive inclusions. For example, one or more of A, B, and C, meaning any of the following: alone A, alone B, alone C, a combination of A and B, a combination of B and C, a combination of A and C, or a combination of A, B, and C.

In addition, “one” or “a” is used to describe elements and components herein. This is only for convenience and to provide a general meaning to the scope of the present disclosure. The description should be understood as including one or at least one, a or at least a, and the singular also includes the plural, unless otherwise clearly indicated.

Unless otherwise defined, the meanings of all technical and scientific terms used in this article are the same as those commonly understood by persons of ordinary skill in the art to which the present disclosure belongs. Although methods and materials similar or equivalent to those described in this article can be used in the practice or testing of the disclosed combination implementation scheme, suitable methods and materials are those described below. Unless specific paragraphs are cited, all publications, patent applications, patents, and other references mentioned herein are incorporated herein by reference in its entirety. In case of any conflict, the specification and its definitions shall prevail. In addition, materials, methods and examples are exemplary, but not limiting.

A protein detection method is a protein detection method based on a Lab-in-Tip technology. The Lab-in-Tip technology is obtained by technical modification on the basis of the pipette sucker of the pipette in the prior art. By utilizing the cone-shaped structure of the pipette sucker, the detection antibody and the SAPE are respectively sealed at different positions of the inner wall of the pipette sucker, the volume of the solvent sucked by the pipette sucker is controlled by the pipette or the pipette workstation to dissolve the detection antibody and the SAPE step by step to enter the detection portion fixed with the probe type graphic encoding chip, thereby achieving the detection of the protein. This method is simple, quick and correct in detection results, the used device is simple and low in cost, and the assembling and disassembling of the detection device are conducted by utilizing the conventional pipette sucker in the lab.

Further, to achieve the above protein detection method, the present disclosure also provides a detection device. The detection device conducts protein quantitative detection by simply modifying the pipette sucker based on the Lab-in-Tip technology. The detection device is simple in structure and easy to assemble. The assembled components are only conventional devices in the lab, such as the pipette sucker, a silicone tube and a capillary tube. Furthermore, the protein detection is simple in process and correct in results, especially, the detection time can be greatly shortened.

Specifically, the detection device comprises a detection tube and a storage tube which are detachably connected; the storage tube and the detection tube are connected through a connection member or directly connected.

As a preferred embodiment, direct connection arrangement comprises: the storage tube and the detection tube are connected through a threaded component, a fastener, a seal or other connection manners, or devices that can achieve the sealing and connection of the storage tube and the detection tube in the prior art are used, which are all included within the scope of protection of the present disclosure.

As a preferred embodiment, the storage tube and the detection tube are connected through the connection member or directly connected.

Further, one end of the connection member is connected with the tip part of the storage tube, and the other end of the connection member is connected with the detection tube.

Further, the connection member is one of latex tube, a rubber tube, a thermoplastic tube or a UV hose, which can seal and connect the storage tube with the detection tube.

Further, the detection tube comprises any one of a capillary tube, a plastic tube, a quartz tube and a glass tube, which fixes the surface modified probe type graphic encoding chip on the inner wall of the storage tube through a natural subsidence method.

Further, the storage tube has a pipette sucker structure; more preferably, the storage tube is in a cone-shaped structure, including a tip part and a tail part; the tip part is connected with the detection tube; the tail part can be connected with the pipette or the pipette workstation. In the embodiments of the present disclosure, the used pipette workstation is an Eppendorf pipette workstation.

Further, the detection antibody and the SAPE are directly lyophilized on the surface of the inner wall of the storage tube through the lyophilization method. The solutions flow through the surface of the inner wall of the storage tube, so as to respectively dissolve the detection antibody and the SAPE.

As a preferred embodiment, the storage portion comprises a first storage area and a second storage area, the first storage area is located at a position between the tip part and ½ of the storage tube, and the detection antibody is sealed on the inner wall of the first storage area.

As a preferred embodiment, the second storage area is located at a position between the tail part and ½ of the storage tube, and the SAPE is sealed on the inner wall of the second storage area.

As another preferred embodiment, the detection antibody and the SAPE are dissolved to be directly prepared into solutions, and then contact and react with the graphic encoding chip in the detection tube in turn under the blowing action of the pipette or the pipette workstation, so as to achieve protein quantitative detection and analysis.

Specifically, the protein detection method combined with the above detection device comprises the following steps:
S1, fixing a probe type graphic encoding chip coupled with a specific capture antibody in the detection device through natural subsidence;
S2, preparing a detection antibody solution and/or an SAPE solution, or allowing the detection antibody and/or SAPE to be embedded in the detection device;
S3, adding a to-be-detected protein sample solution into a sample well plate, and putting the well plate in the pipette workstation;
S4, connecting a Lab-in-Tip device with a pipette workstation so that the Lab-in-Tip device conducts blowing in the well plate; washing after the blowing is ended;
S5, putting a well plate distributed with a phosphate buffer solution into the pipette workstation so that the Lab-in-Tip device conducts blowing in the well plate again, first, dissolving the sealed detection antibody to react with the graphic encoding chip; after the reaction is ended, washing, or placing the prepared detection antibody solution in the well plate so that the detection antibody solution directly reacts with the graphic encoding chip;
S6, repeating the operation in the above step, putting the prepared SAPE solution into the detection device, or, dissolving the sealed SAPE again so that SAPE reacts with the graphic encoding chip; and washing after the reaction is ended; and
S7, taking out the detection tube for image data collection, imaging in an optical channel with a set wavelength to perform qualitative analysis or quantitative analysis on a target substance in a liquid system therein to obtain determination results.

As a preferred embodiment, the probe type graphic encoding chip comprises: after surface modification is performed on the graphic encoding chip, a probe molecule is coupled to the graphic encoding chip to obtain a probe type graphic encoding chip; the probe molecule is a specific capture antibody; more preferably, the graphic encoding chip is an encoding suspension chip based on silica particles, and can provide a 128-plexed encoding space.

As a preferred embodiment, a method for preparing the probe type graphic encoding chip comprises the following steps:
(1) dispersing an encoding suspension chip based on silica into a solution of amino polydimethylsiloxane (APDMS) in ethanol for reaction, so that the encoding suspension chip is subjected to amino surface modification to obtain the amino modified encoding suspension chip;
(2) dispersing the amino modified encoding suspension chip obtained after reaction in step (1) into a succinic anhydride solution, and then performing vibration reaction at a room temperature for surface modification, so as to obtain the carboxyl modified graphic encoding chip; and
(3) performing an activation reaction on the surface modified encoding chip obtained in step (2), and then performing coupling reaction on the activated encoding chip and a probe molecule solution to couple the probe molecule to the surface of the graphic encoding chip so as to obtain the probe type graphic encoding chip.

Alternatively, the modification and detection of the above probe type graphic encoding chip can also refer to relevant technical solutions disclosed in Chinese invention patent CN114965397A.

Next, the technical solution of the present disclosure, its implementation process and principle and the like will be further explained through specific embodiments. It should be noted that the following detailed descriptions are exemplary, and intended to further explain the present disclosure. The described embodiments are only some embodiments of the present disclosure, but not all the embodiments. Based on the embodiments of the present disclosure, embodiments obtained by persons of ordinary skill in the art without creative efforts are all included within the scope of protection of the present disclosure. Unless otherwise specified, reagents and raw materials used in the following examples are commercially available, and test methods without specific conditions are usually conducted under conventional conditions or according to the conditions recommended by each manufacturer. Also, unless otherwise specified, experimental methods, detection methods and preparation methods disclosed in the present disclosure all adopt conventional technologies in the art. These technologies have been completely described in literatures.

### EXAMPLE 1

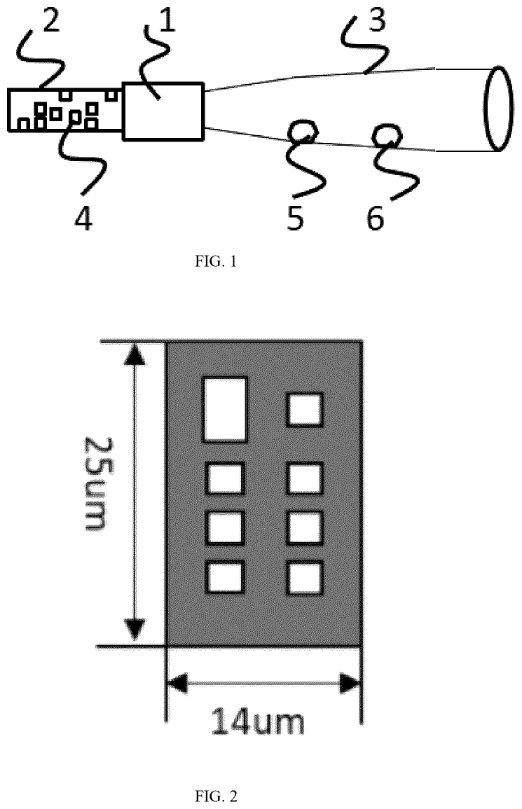

This example provides a protein detection device based on a Lab-in-Tip technology. The specific structure is seen in FIG. 1.

As shown in FIG. 1, the protein detection device comprised a silicone tube 1, a capillary tube 2 and a pipette sucker 3. The capillary tube 2 and the pipette sucker 3 were respectively connected at two ends of the silicone tube 1, the sizes of the connection parts of the silicone tube 1, the capillary tube 2 and the pipette sucker 3 were matched so as to ensure that the whole connected protein detection device could not leak.

A probe type graphic encoding chip 4 coupled with different capture antibodies was fixed on the inner wall of the capillary tube 2.

Specifically, the pipette sucker 3 was in a cone-shaped structure, the inner wall of the pipette sucker comprised a first storage area 5 and a second storage area 6, the first storage area 5 was arranged at one end connected with the silicone tube 1, that is, a region between one end close to the tip part of the pipette sucker 3 and the ½ of the middle of the pipette sucker 3.

The second storage area 6 was arranged on the tail part, that is, a region between a port close to the pipette workstation and the ½ of the middle of the pipette sucker 3.

In this example, a biotinylated detection antibody was fixed in the first storage area 5, and SAPE (fluorescently labeled streptavidin) was fixed in the second storage area 6.

Specifically, a fixing method was that the detection antibody and the SAPE were directly fixed in the pipette sucker 3 respectively through a lyophilization method.

The lyophilization method comprised: the detection antibody was placed in the first storage 5 and SAPE was placed in the second storage 6, followed by freezing the pipette sucker 3 in a refrigerator at −80° C. for 20 min, and the pipette sucker 3 was lyophilized for 1.5 h in a lyophilizer after the detection antibody and the SAPE become solids.

Further, a preparation method of a probe type graphic encoding chip 4 comprised:
(1) an encoding suspension chip based on silica was provided and 2×10⁵ encoding suspension chips were selected to be dispersed into 1000 μL of 5% amino polydimethylsiloxane ethanol solution (prepared with 95% ethanol), and washed after sufficient reaction for 30 min;
(2) supernatant was discarded and then the suspension chip was dispersed into 1000 μL of 10% succinic anhydride solution, an oscillation reaction was performed at a room temperature overnight, and then washed, so as to obtain a carboxyl chip; and
(3) an activating solution containing 130 mmol/L EDC and 326 mmol/L NHS using a 0.1 mol/L MES buffer solution (pH=4.7) was prepared, allowing the chip suspension solution and the activating solution to be mixed and react for about 30 min followed by washing, subsequently mixing and reacting the washed reaction product with a probe molecule solution (a solvent was a 0.1 mol/L NaAc-HAc buffer solution) at 4° C. for 6 h-12 h, so as to obtain a probe type graphic encoding chip, wherein the probe in this example was a capture antibody.

Referring to FIG. 2, which is a probe type graphic encoding chip prepared in this exampl, the chip had a size of 14×25 μm and a 128-plexed encoding space, and could provide a user with 128-plexed detection.

The probe type graphic encoding chip was embedded in the inner all of the capillary tube 2 through a natural subsidence method, and the silicone tube 1, the capillary tube 2 and the pipette sucker 3 were respectively connected to be assembled, so as to obtain a Lab-in-Tip detection device.

Protein detection was carried out based on the above Lab-in-Tip detection device. The specific detection method comprised the following steps:
a, the graphic encoding chips were precisely manufactured by a photolithography technique and released and then underwent carboxyl modification, subsequently different types of specific capture antibodies were coated on different graphic encoding chips to obtain different encoding chips coupled with different types of capture antibodies, in this example, the capture antibodies were named as Purified anti-human IL-8 and Purified anti-human IL-1β;
b, the different encoding chips coupled with different types of capture antibodies were fixed on the inner wall of the capillary tube 2;
c, a mixture of biotinylated detection antibodies at a required concentration and SAPE were prepared and then sealed at front and back ends of the pipette sucker 3, the initial concentration of the detection antibody was 35 μg/mL, the sampling volume of the detection antibody was 2 μL, the final concentration of the detection antibody after dissolution was 1 μg/mL, the concentration of SAPE was 30 μg/mL, the sampling volume of SAPE was 5 μL, and the final concentration of SAPE was 0.5 μg/mL;
d, the silicone tube 1 was connected with a square capillary tube 2 and the pipette sucker 3 in turn;
e, 50 μL of sample solution was added into each well of a 96-well plate, and the 96-well plate was put in a pipette workstation;
f, the detection device based on Lab-in-Tip was inserted into a machine sucker in the pipette workstation, and the instrument was turned on, so that the detection device based on Lab-in-Tip conducted self blowing for 30 min in sample wells;
g, after the above blowing was ended, in the pipette workstation, the detection device based on Lab-in-Tip automatically rotated to a washing tank to up and down blow 3 times for washing;
h, 70 μL of 1×phosphate buffer solution was allocated to each well of a newly prepared 96-well plate, so that the detection device based on Lab-in-Tip conducted self blowing for 20 min in this well, the volume of the phosphate buffer saline entering the pipette sucker did not excess the ½ position of the pipette sucker, in such the way, the detection antibody sealed in the inner wall of the pipette sucker 3 in advance was blown and dissolved, and SAPE sealed in the second storage portion 6 was not affected by the phosphate buffer solution, the final concentration of the antibody obtained after dissolution was 1 μg/mL, and then the antibody was allowed to enter the capillary tube 2 to react with the specific capture antibody on the surface of the multi-encoding chip in the inner wall of the capillary tube 2;
i, after the above blowing was ended, the detection device based on Lab-in-Tip automatically rotated to the washing tank again to up and down blow 3 times for washing;
j, 120 μL of 1×phosphate buffer solution was allocated to each well of a newly prepared 96-well plate, so that the detection device based on Lab-in-Tip conducted self blowing for 10 min in this well, SAPE sealed in advance was blown and dissolved, the volume of the phosphate buffer solution entering the pipette sucker exceeds the position of the second storage so as to ensure SAPE could be completely dissolved, the final concentration of SAPE obtained after dissolution was 0.5 μg/mL, and SAPE reacted with the specific antibody on the surface of the multi-encoding chip in the inner wall of the capillary tube 2;
k, after the above blowing was ended, the detection device based on Lab-in-Tip automatically rotated to the washing tank again to up and down blow 5 times for washing;
l, the capillary tube 2 was taken out and directly used for image data collection to perform qualitative analysis or quantitative analysis treatment to obtain determination results.

In this example, the sample solution used human cytokine IL-8 as a model protein analyte, and the analyte was dissolved into the buffer solution; the initial concentration of the sample solution was 10 ng/ml; after the sample solution was diluted by 5 folds, the 7th concentration was 0.64 pg/mL.

Analysis was conducted through the above detection method. The specific encoding chip in the detection tube was attached with the specific capture antibody to capture a to-be-detected substance in the sample. The quantities of multiple soluble components in the sample were determined and analyzed according to different fluorescence intensities carried on different encoding chips.

Referring to FIG. 3, which is a standard curve graph showing the detection performance of human cytokine IL-8 (purchased from R&D) in a reaction time of 1 h. In this figure, a dotted line at the bottom is blank (protein content is 0), which is a detection line which does not contain the analyte. By analysis, it can be seen that by using the technical solution of the present disclosure, the sensitivity of protein quantitative analysis is as high as pg/mL level (there is still obvious distinction between chip signal values near a protein concentration of 1 pg/mL). A target protein is quantified by correlating the fluorescence intensity of the sample with the standard curve.

### EXAMPLE 2

This example is different from example 1 in that the to-be-detected samples are 13 human cytokines as model protein analytes. The human cytokines are IL-1β, IL-2, IL-4, IL-5, IL-6, IL-8, IL-10, IL-12, IL-17, TNF-α, IFN-α, IFN-≢ and GM-CSF, respectively (all of which are purchased from R&D).

13 protein analytes were mixed, and 13 detection antibodies were mixed in advance and sealed in a Lab-in-Tip sucker. Through a Lab-in-Tip technology, specific operation steps are the same as those in example 1. Furthermore, the above 13-plexed detection was completed within the reaction time of 1 h in this example.

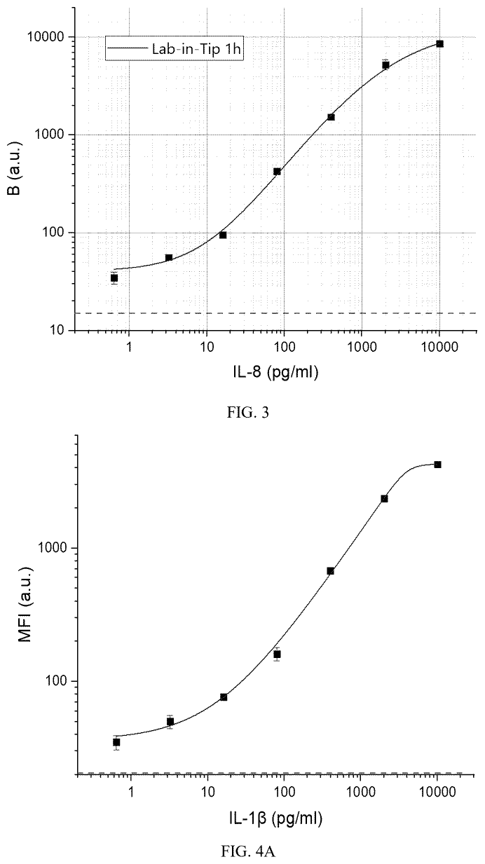

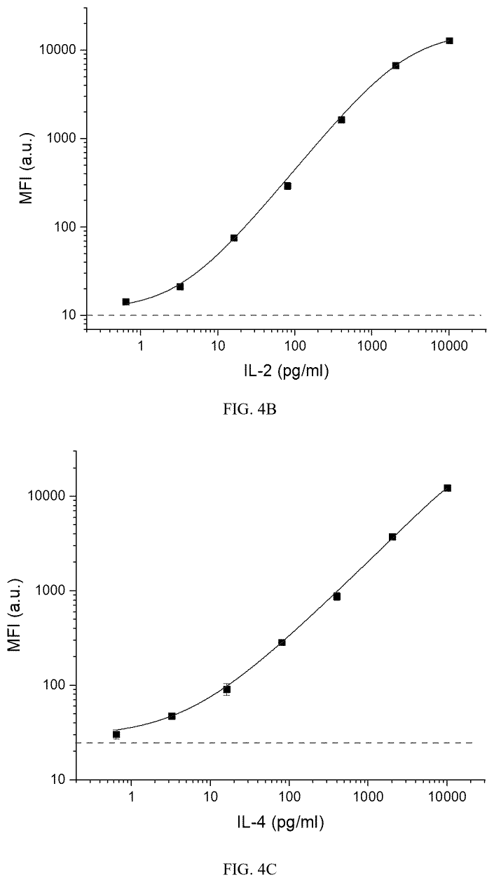

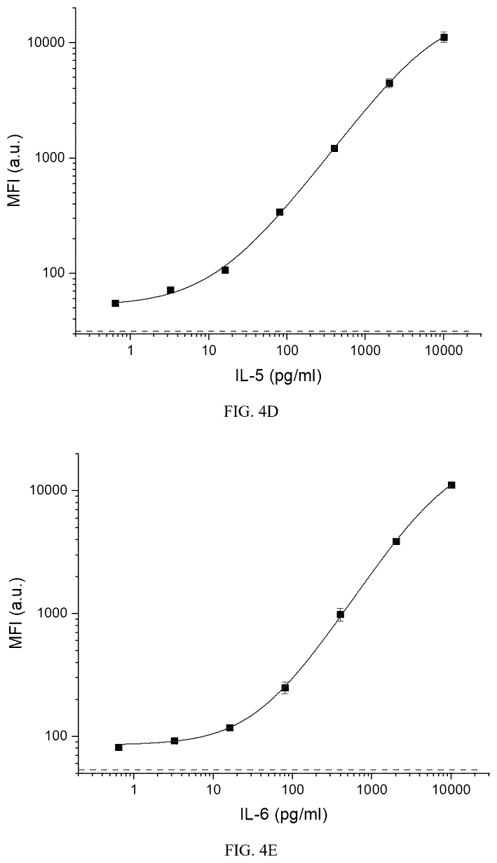

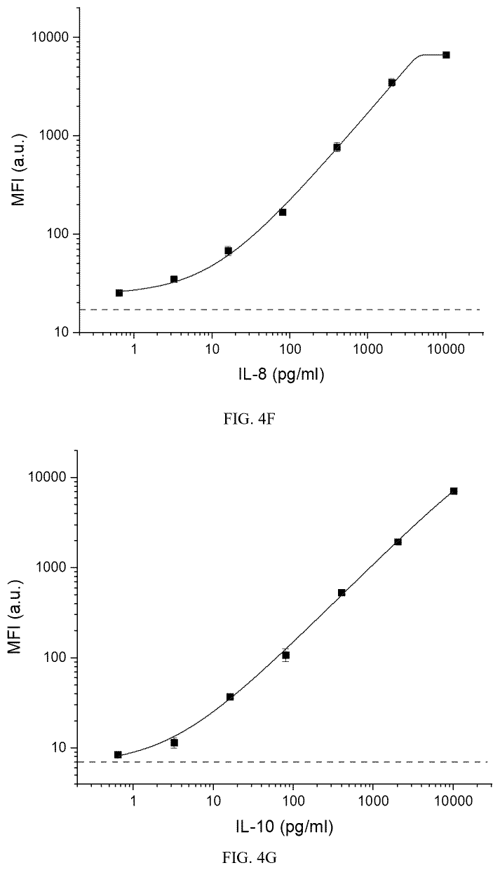

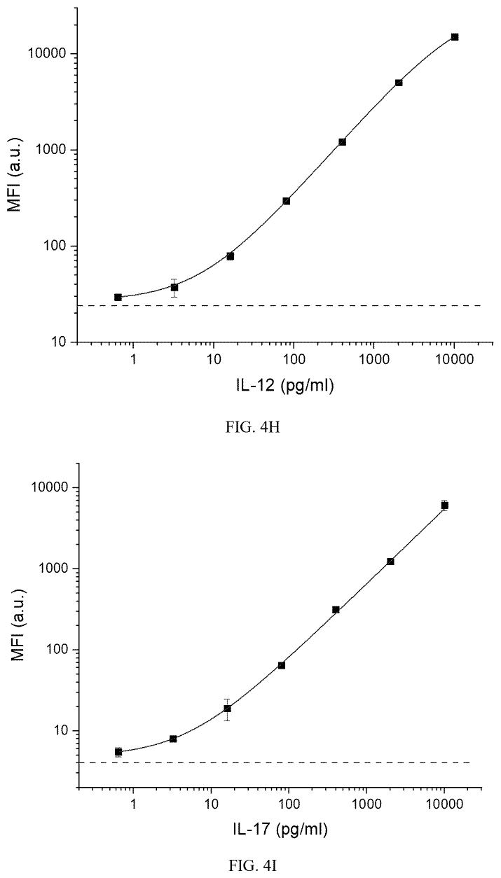

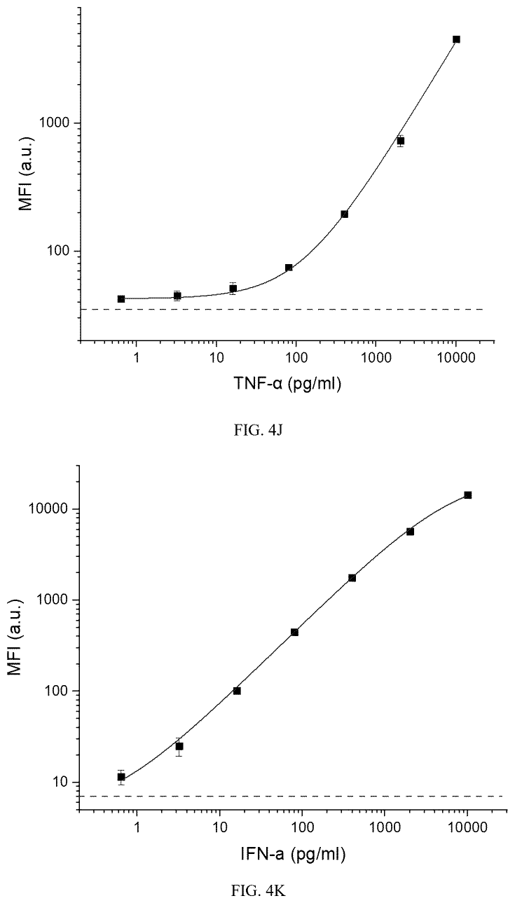

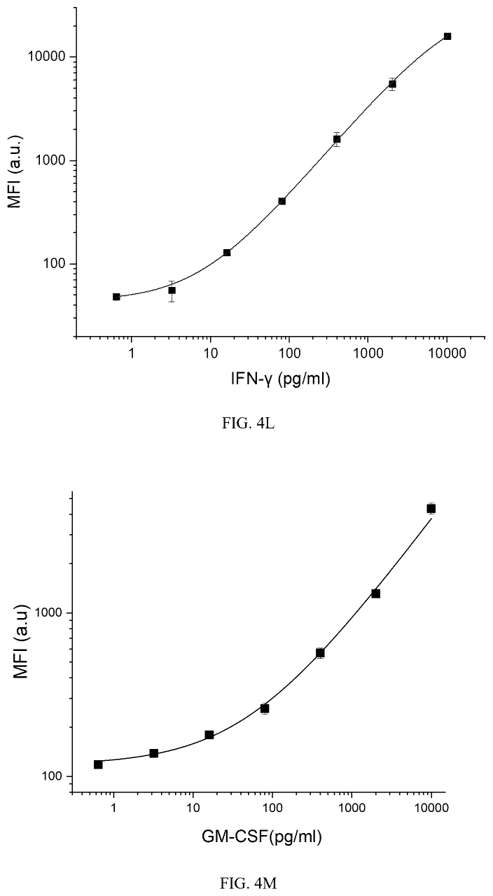

The detection results are seen in FIGS. 4A-4M. It can be seen from standard curves that the sensitivity of protein quantitative analysis is as high as pg/mL level, and there is still obvious distinction between chip signal values near a protein concentration of 1 pg/mL.

### EXAMPLE 3

This example is the same as example 1 except that the assembled Lab-in-Tip detection device in this example is stored for 3 months in a refrigerator at 4° C. and then detection is conducted.

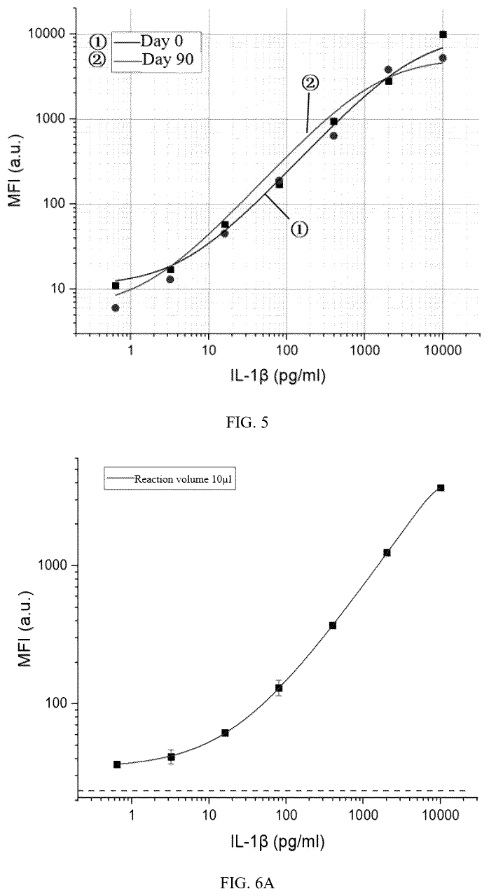

The detection results are seen in FIG. 5. The sensitivity of protein quantitative analysis still maintains pg/mL level, specifically, a storage signal value floats up or down in a 20% error range without obvious reduction. Meanwhile, the IL-1β detection sensitivity detected in the method can reach 1 pg/mL.

### EXAMPLE 4

This example is different from example 1 that to-be-detected samples are 4 human cytokines as model protein analytes. The human cytokines are IL-4, IL-6, IL-8 and IL-1β (all of which are purchased from R&D). 4-plexed detection is completed by using 10 μL of samples.

4 protein analytes are mixed, and 4 detection antibodies are mixed in advance and sealed in a Lab-in-Tip sucker. Through the Lab-in-Tip technology, 4-plexed detection is completed by only using 10 μL of samples (the volume of the sample in example 1 is 50 μL) within the reaction time of 1 h.

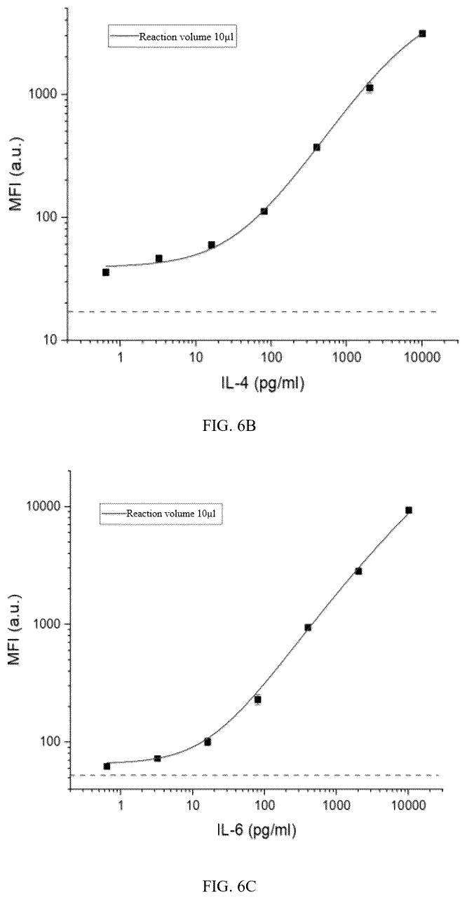

The detection results are seen in FIGS. 6A-6D. It can be seen from standard curves that there is still obvious distinction between chip signal values near a protein concentration of 1 pg/mL.

### EXAMPLE 5

This example is different from example 1 that to-be-detected samples are 3 human cytokines as model protein analytes. The human cytokines are IL-4, IL-6 and IL-8 (all of which are purchased from R&D). 3-plexed detection is completed within the reaction time of 15 min.

3 protein analytes were mixed, and 3 detection antibodies were mixed in advance and sealed in a Lab-in-Tip sucker. Through the Lab-in-Tip technology, 3-plexed detection was completed within the reaction time of only 15 min (the reaction time is 1 h in example 1).

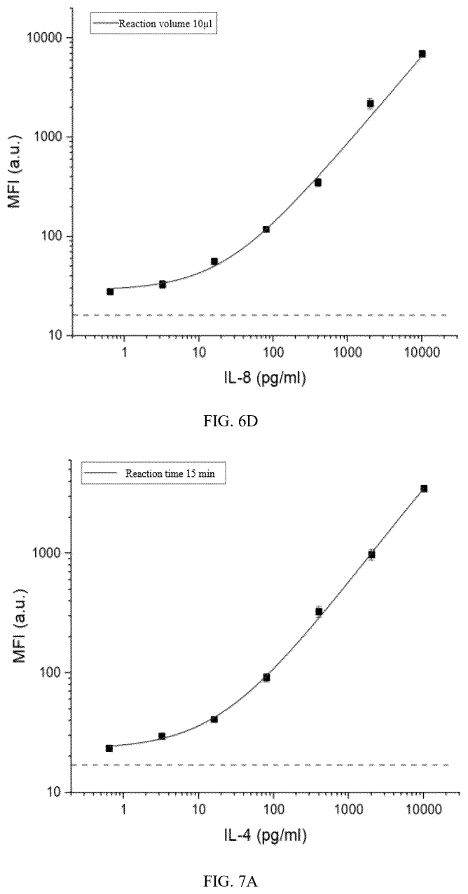

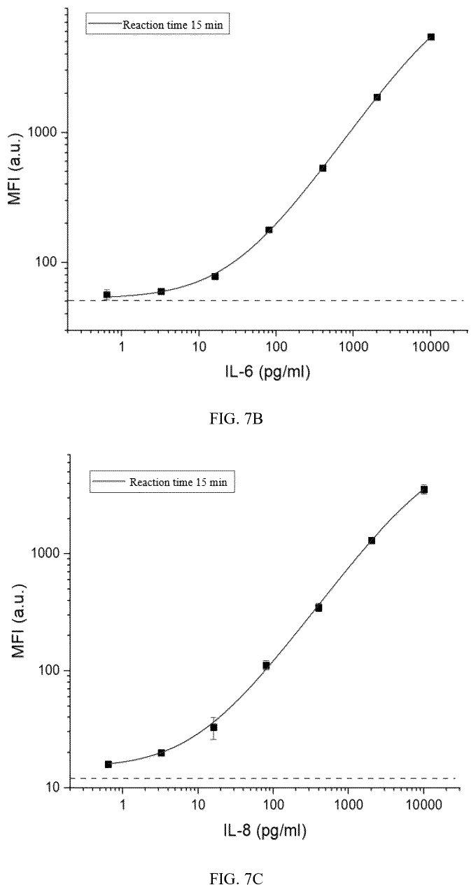

The detection results are seen in FIGS. 7A-7C. It can be seen from standard curves that protein quantitative analysis sensitivity is as high as pg/mL level (there is still obvious distinction between chip signal values near a protein concentration of 1 pg/mL).

Through detection results in examples 1-5, it can be seen that the protein concentration can be detected as 1 pg/mL level, there is also still obvious distinction between chip signal values near a protein concentration of 1 pg/mL (a concentration of 1 pg/ml), and the signal values at this point are all higher than a background signal value, and multiplexed detections can be completed, superlatively, 128 detections can be achieved by using the Lab-in-Tip detection device provided by the present disclosure.

Furthermore, in example 4, only 10 μL of detection sample is needed to complete high-sensitivity (pg/mL level) detection and analysis, and there is obvious distinction between chip signal values.

In example 5, 3-plexed detection is completed within 15 min, indicating that quick detection can be achieved by using the method of the present disclosure.

By using the Lab-in-Tip detection device provided by the present disclosure, the graphic encoding chip can be fixed on the inner wall of the capillary tube and assembled into a structure of a biologically common pipette sucker; meanwhile, biotinylated detection antibodies and proteins such as SAPE are sealed inside the pipette sucker in advance, so that protein quantitative analysis based on ELISA principle is completed within a short period of time through one Lab-in-Tip detection device having an improved pipette tip structure common in the prior art; and a sensitivity of 1 pg/mL can be achieved within the reaction time of 15 min.

Especially, in actual detection, high-sensitivity detection can be achieved by only selecting and assembling detection devices conventionally used in a lab without special detection devices.

Obviously, the present disclosure is based on the Lab-in-Tip technology, the used graphic encoding chip provides a 128-plexed encoding space, so as to complete 128-plexed detection in a single pipette sucker, thereby greatly reducing the sample size required in the reaction process, significantly shortening the time of washing steps, and performing a multiplexed detection which is quick, convenient, high in throughput, few in sample size, good in repeatability, high in sensitivity and wide in linear range.

The above descriptions are only preferred embodiments of the present disclosure, but do not limit the protective scope of the present disclosure. For those skilled in the art, various changes and variations can be made to the present disclosure. Within the spirit and principle of the present disclosure, through the conventional replacement or the same functions, changes, modifications, replacements, integrations and parameter changes made to these embodiments without departing from the principle and spirit of the present disclosure are all included within the scope of protection of the present disclosure.

What is claimed is:

1. A detection device, applied in a protein detection method for a protein qualitative or quantitative detection, wherein the detection device comprises a detection tube and a storage tube that are detachably connected, and the protein detection method comprises: fixing a probe type graphic encoding chip coupled with a specific capture antibody in the detection device, meanwhile respectively sealing R-phycoerythrin labeled streptavidin (SAPE) and a detection antibody in the detection device, or preparing an SAPE solution and a detection antibody solution in advance; and
during a detection, respectively dissolving the detection antibody and the SAPE into the detection device, or transferring the SAPE solution and the detection antibody solution which are prepared in advance into the detection device, and then performing the detection and an analysis through the probe type graphic encoding chip so as to obtain protein quantitative analysis results.

2. The detection device according to claim 1, wherein the probe type graphic encoding chip comprises: performing a surface modification on a graphic encoding chip, followed by coupling a probe molecule to the graphic encoding chip so as to obtain the probe type graphic encoding chip;
and/or, the probe molecule is the specific capture antibody;
and/or, the graphic encoding chip is an encoding suspension chip based on silica particles.

3. The detection device according to claim 2, wherein a preparation method of the probe type graphic encoding chip comprises the following steps:
(1) dispersing the encoding suspension chip based on the silica particles into an ethanol solution of aminosilane for a reaction, so that the encoding suspension chip is subjected to an amino surface modification to obtain an amino modified encoding suspension chip;
(2) dispersing the amino modified encoding suspension chip obtained after the reaction in the step (1) into a succinic anhydride solution, and then performing a vibration reaction at a room temperature for the surface modification, so as to obtain a carboxyl modified graphic encoding chip; and
(3) performing an activation reaction on the carboxyl modified graphic encoding chip obtained in the step (2), and then performing a coupling reaction on an activated graphic encoding chip and a probe molecule solution to couple the probe molecule to a surface of the activated graphic encoding chip so as to obtain the probe type graphic encoding chip.

4. The detection device according to claim 3, wherein in the step (3), the activation reaction is carried out at the room temperature for 20-40 min; an activation solution is a 4-methylmorpholine sulfonic acid (MES) buffer solution containing 1-ethyl (3-dimethylaminopropyl) carbodiimide (EDC) and N-hydroxysuccinimide (NHS)

5. The detection device according to claim 3, wherein in the step (3), the coupling reaction is carried out at 0-4° C. for 6 h-12 h;
and/or, the probe molecule solution is a NaAc-HAc buffer solution of the probe molecule.

6. The protein detection method device according to claim 1, wherein the probe type graphic encoding chip coupled with the specific capture antibody is embedded in the detection device based on a Lab-in-Tip technology; the detection antibody solution and the SAPE solution are respectively put in the detection device in turn so as to achieve a protein quantitative detection and analysis through the probe type graphic encoding chip;
preferably, the detection device at least comprises the detection tube and the storage tube, and the probe type graphic encoding chip is embedded in the detection tube;
the detection antibody and the SAPE are embedded in an inner wall of the storage tube, and then respectively enter the detection tube after blowing and dissolving via a pipette workstation to react with the probe type graphic encoding chip; or, the detection antibody solution and the SAPE solution prepared in advance are directly put into the detection tube in turn to react with the probe type graphic encoding chip.

7. The detection device according to claim 1, wherein the protein detection method comprises the following steps:
S1, fixing the probe type graphic encoding chip coupled with the specific capture antibody in the detection device through natural subsidence;
S2, preparing the detection antibody solution and/or the SAPE solution, or embedding the detection antibody and/or the SAPE in the detection device;
S3, adding a to-be-detected protein sample solution into a sample well plate, and putting the sample well plate in a pipette workstation;
S4, connecting the detection device with the pipette workstation or a pipette;
S5, putting a well plate distributed with a phosphate buffer solution into the pipette workstation, dissolving the detection antibody embedded in the detection device to form a solution, or, placing the prepared detection antibody solution into the well plate, and performing a solution blowing action through the pipette or the pipette workstation so that the detection antibody solution reacts with the probe type graphic encoding chip; and washing after a first reaction is ended;
S6, repeating an operation in the above step, putting the prepared SAPE solution into the detection device, or, dissolving the SAPE embedded in the detection device so that the SAPE reacts with the probe type graphic encoding chip; and washing after a second reaction is ended; and
S7, taking out the detection tube for an image data collection, and performing a qualitative analysis or quantitative analysis treatment on the probe type graphic encoding chip in the detection tube to obtain determination results.

8. (canceled)

9. The detection device according to claim 1, wherein the storage tube has a pipette sucker structure based on a Lab-in-Tip technology.

10. The detection device according to claim 1, wherein the storage tube is in a cone-shaped structure, and comprises a tip part and a tail part; the tip part is connected with the detection tube; the tail part is connected with a pipette or a pipette workstation;
the probe type graphic encoding chip is fixed in the detection device; and
the detection antibody and/or the SAPE enter the detection tube to react with the probe type graphic encoding chip.

11. The detection device according to claim 10, wherein the detection antibody and the SAPE are sealed at different positions of a surface of an inner wall of the storage tube, and then respectively enter the detection tube after being dissolved; preferably, the detection antibody is sealed on the surface of the inner wall of an end of the storage tube close to the detection tube, and the SAPE is located at a tail part of a cone-shaped sucker, a rear end of the cone-shaped sucker is close to the storage tube;
or, the detection antibody and the SAPE are dissolved to be prepared into the solutions, and then contact and react with the probe type graphic encoding chip in the detection tube in turn under a blowing action of the pipette or the pipette workstation, so as to achieve a protein quantitative detection and analysis.

12. The detection device according to claim 11, wherein the detection antibody and the SAPE are respectively stored in the storage tube by using a lyophilization method, or the detection antibody solution and the SAPE solution are respectively added in the storage tube, then enter the detection tube after being dissolved and react with the specific capture antibody coupled with a surface of the probe type graphic encoding chip;
a storage portion comprises a first storage area and a second storage area;
the first storage area is located between the tip part and ½ of the storage tube, and the detection antibody is sealed on an inner wall of the first storage area; and
the second storage area is located between the tail part and ½ of the storage tube, and the SAPE is sealed on an inner wall of the second storage area.

13. The detection device according to any one of claim 1, wherein the storage tube and the detection tube are connected through a connection member, or directly connected;
a first end of the connection member is connected with a tip part of the storage tube, and a second end of the connection member is connected with the detection tube; and
the connection member is one of a latex tube, a rubber tube, a thermoplastic tube, or a ultraviolet (UV) hose, the connection member seals and connects the storage tube and the detection tube.

14. The detection device according to claim 11, wherein the detection tube comprises any one of a capillary tube, a plastic tube, a quartz tube, and a glass tube, the detection tube fixes a surface modified probe type graphic encoding chip on the inner wall of the storage tube through a natural subsidence method.

15. (canceled)

16. The detection device according to claim 2, wherein the probe type graphic encoding chip coupled with the specific capture antibody is embedded in the detection device based on a Lab-in-Tip technology; the detection antibody solution and the SAPE solution are respectively put in the detection device in turn so as to achieve a protein quantitative detection and analysis through the probe type graphic encoding chip;
preferably, the detection device at least comprises the detection tube and the storage tube, and the probe type graphic encoding chip is embedded in the detection tube;
the detection antibody and the SAPE are embedded in an inner wall of the storage tube, and then respectively enter the detection tube after blowing and dissolving via a pipette workstation to react with the probe type graphic encoding chip; or, the detection antibody solution and the SAPE solution prepared in advance are directly put into the detection tube in turn to react with the probe type graphic encoding chip.

17. The detection device according to claim 3, wherein the probe type graphic encoding chip coupled with the specific capture antibody is embedded in the detection device based on a Lab-in-Tip technology; the detection antibody solution and the SAPE solution are respectively put in the detection device in turn so as to achieve a protein quantitative detection and analysis through the probe type graphic encoding chip;
preferably, the detection device at least comprises the detection tube and the storage tube, and the probe type graphic encoding chip is embedded in the detection tube;
the detection antibody and the SAPE are embedded in an inner wall of the storage tube, and then respectively enter the detection tube after blowing and dissolving via a pipette workstation to react with the probe type graphic encoding chip; or, the detection antibody solution and the SAPE solution prepared in advance are directly put into the detection tube in turn to react with the probe type graphic encoding chip.

18. The detection device according to claim 4, wherein the probe type graphic encoding chip coupled with the specific capture antibody is embedded in the detection device based on a Lab-in-Tip technology; the detection antibody solution and the SAPE solution are respectively put in the detection device in turn so as to achieve a protein quantitative detection and analysis through the probe type graphic encoding chip;
preferably, the detection device at least comprises the detection tube and the storage tube, and the probe type graphic encoding chip is embedded in the detection tube;
the detection antibody and the SAPE are embedded in an inner wall of the storage tube, and then respectively enter the detection tube after blowing and dissolving via a pipette workstation to react with the probe type graphic encoding chip; or, the detection antibody solution and the SAPE solution prepared in advance are directly put into the detection tube in turn to react with the probe type graphic encoding chip.

19. The detection device according to claim 5, wherein the probe type graphic encoding chip coupled with the specific capture antibody is embedded in the detection device based on a Lab-in-Tip technology; the detection antibody solution and the SAPE solution are respectively put in the detection device in turn so as to achieve a protein quantitative detection and analysis through the probe type graphic encoding chip;
preferably, the detection device at least comprises the detection tube and the storage tube, and the probe type graphic encoding chip is embedded in the detection tube;
the detection antibody and the SAPE are embedded in an inner wall of the storage tube, and then respectively enter the detection tube after blowing and dissolving via a pipette workstation to react with the probe type graphic encoding chip; or, the detection antibody solution and the SAPE solution prepared in advance are directly put into the detection tube in turn to react with the probe type graphic encoding chip.

20. The detection device according to claim 2, wherein the protein detection method comprises the following steps:
S1, fixing the probe type graphic encoding chip coupled with the specific capture antibody in the detection device through natural subsidence;
S2, preparing the detection antibody solution and/or the SAPE solution, or embedding the detection antibody and/or the SAPE in the detection device;
S3, adding a to-be-detected protein sample solution into a sample well plate, and putting the sample well plate in a pipette workstation;
S4, connecting the detection device with the pipette workstation or a pipette;
S5, putting a well plate distributed with a phosphate buffer solution into the pipette workstation, dissolving the detection antibody embedded in the detection device to form a solution, or, placing the prepared detection antibody solution into the well plate, and performing a solution blowing action through the pipette or the pipette workstation so that the detection antibody solution reacts with the probe type graphic encoding chip; and washing after a first reaction is ended;
S6, repeating an operation in the above step, putting the prepared SAPE solution into the detection device, or, dissolving the SAPE embedded in the detection device so that the SAPE reacts with the probe type graphic encoding chip; and washing after a second reaction is ended; and
S7, taking out the detection tube for an image data collection, and performing a qualitative analysis or quantitative analysis treatment on the probe type graphic encoding chip in the detection tube to obtain determination results.

21. The detection device according to claim 3, wherein the protein detection method comprises the following steps:
S1, fixing the probe type graphic encoding chip coupled with the specific capture antibody in the detection device through natural subsidence;
S2, preparing the detection antibody solution and/or the SAPE solution, or embedding the detection antibody and/or the SAPE in the detection device;
S3, adding a to-be-detected protein sample solution into a sample well plate, and putting the sample well plate in a pipette workstation;
S4, connecting the detection device with the pipette workstation or a pipette;
S5, putting a well plate distributed with a phosphate buffer solution into the pipette workstation, dissolving the detection antibody embedded in the detection device to form a solution, or, placing the prepared detection antibody solution into the well plate, and performing a solution blowing action through the pipette or the pipette workstation so that the detection antibody solution reacts with the probe type graphic encoding chip; and washing after a first reaction is ended;
S6, repeating an operation in the above step, putting the prepared SAPE solution into the detection device, or, dissolving the SAPE embedded in the detection device so that the SAPE reacts with the probe type graphic encoding chip; and washing after a second reaction is ended; and
S7, taking out the detection tube for an image data collection, and performing a qualitative analysis or quantitative analysis treatment on the probe type graphic encoding chip in the detection tube to obtain determination results.

22. The detection device according to claim 4, wherein the protein detection method comprises the following steps:
S1, fixing the probe type graphic encoding chip coupled with the specific capture antibody in the detection device through natural subsidence;
S2, preparing the detection antibody solution and/or the SAPE solution, or embedding the detection antibody and/or the SAPE in the detection device;
S3, adding a to-be-detected protein sample solution into a sample well plate, and putting the sample well plate in a pipette workstation;
S4, connecting the detection device with the pipette workstation or a pipette;
S5, putting a well plate distributed with a phosphate buffer solution into the pipette workstation, dissolving the detection antibody embedded in the detection device to form a solution, or, placing the prepared detection antibody solution into the well plate, and performing a solution blowing action through the pipette or the pipette workstation so that the detection antibody solution reacts with the probe type graphic encoding chip; and washing after a first reaction is ended;
S6, repeating an operation in the above step, putting the prepared SAPE solution into the detection device, or, dissolving the SAPE embedded in the detection device so that the SAPE reacts with the probe type graphic encoding chip; and washing after a second reaction is ended; and
S7, taking out the detection tube for an image data collection, and performing a qualitative analysis or quantitative analysis treatment on the probe type graphic encoding chip in the detection tube to obtain determination results.
